(19) United States
(12) Patent Application Publication
(10) Pub. No.: US 2003/0186847 A1
(43) Pub. Date: Oct. 2, 2003

(54) Insulin derivatives and synthesis thereof

(76) Inventors: Richard Henry Jones, London (GB); Fariba Shojaee-Moradi, London (GB); Dietrich Brandenburg, Reichelsheim (DE); Erik Sundermann, Hofheim A T (DE)

Correspondence Address: NIXON & VANDERHYE, PC, 1100 N GLEBE ROAD, 8TH FLOOR, ARLINGTON, VA, 22201-4714, US

(21) Appl. No.: 10/332,157

(22) PCT Filed: Jul. 10, 2001
(86) PCT No.: PCT/GB01/03071

(30) Foreign Application Priority Data
Jul. 10, 2000 (EP) 00305809.6

(51) Int. Cl. A61K 38/28; C07K 14/62

(52) U.S. Cl. 514/3; 530/303

(57) ABSTRACT

Derivatives of insulin are described which are conjugated to thyroid hormones. The thyroid hormone is, for instance, D-thyroxine (3,3′,5,5′-tetraiodo-D-thyronine). Other analogues are described in which a spacer having a alkanediyl chain at least eleven carbon atoms long is included. Binding studies show useful binding characteristics to thyroid binding proteins. New synthetic methods in which racemisation of the thyroxin is minimised, are described.

The present invention relates to insulin derivatives and their synthesis. More specifically insulin is conjugated through the B1 residue (phenylalanine) by conjugating the free amine group to a thyroid hormone via a peptide bond.

In WO-A-95/05187 insulin derivatives are described which have bound thereto a molecular moiety which has an affinity to circulating binding protein. The molecular moiety specifically described and exemplified in that specification was thyroid hormone, specifically L-thyroxine (3,3′,5,5′-tetraiodo-L-thyronine). The covalent conjugation of the thyronine compound to insulin was through peptide bond formation between the free alpha amino group of the B1 residue of insulin to the carboxyl group of the thyronine compound. It has been shown that the L-thyroxine derivative of insulin has affinity to specific plasma proteins, specifically thyroid binding globulin and transthyretin. The binding of the thyronine moiety leads to an altered distribution of insulin, and in particular is believed to render the insulin hepatoselective.

It was found, however, that the L-thyroxine derivative (LT4-Ins) had a very high affinity towards plasma proteins and exhibited limited metabolic turnover. Derivatives having lower affinity for binding proteins have been described in WO-A-99/65941; a further thyroid derivative of insulin is described, namely 3,3′,5′-triiodothyronine, reverse T3-insulin (rT3-Ins).

In WOA-95/07931, insulin is derivatised by reacting the epsilon-amino group of the B29 lysine moiety with L-thyroxine and D-thyroxine, optionally with a C10 spacer. In some examples the amine group of the thyronine moiety is acetylated prior to conjugation of the T4 reagent with insulin.

The binding of thyroid hormones to endogenous circulating proteins is summarised by Robbins, J. et al in Thyroid Hormone Metabolism (ed Hennemann, G.) 1986, Marcel Dekker, NC. USA, 3 to 38. The relative binding affinities of various thyroid hormones is discussed including LT4, T3(3,3′,5-triiodothyronine), rT3,3′,5′-diiodothyronine (3′,5′T2), DT4, N-acetylated LT4, N-acetylated T3 and other alkanoated compounds to thyroid hormone binding proteins (THBPS) such as thyroxine binding globulin (TBG), prealbumin (also known as transthyretin) and albumin.

It would be desirable to optimise the thyroid hormone moiety in insulin conjugates, and its mode of conjugation to insulin, to achieve optimum distribution of insulin within the body, metabolic availability and minimise side effects due to activity of the thyroid hormone moieties.

According to a first aspect of the present invention there is provided a novel compound consisting of insulin or a functional equivalent thereof having covalently bound to the alpha-amine group of the B1 residue a 3,3′,5,5′-tetraiodo-D-thyroxyl group.

The thyroxyl group, known hereinafter as a DT4-yl group, may be bound directly to the alpha amine group through a peptide bond with the carboxyl group of the T4 molecule. Alternatively, there may be a linker provided between the amine group and the carboxyl group. Preferably the linker is joined through peptide bonds at each end to the respective moieties, and has an alkane-diyl group, for instance at least eleven carbon atoms long between the two peptide bonds. Alternatively a shorter linker may be used. Other means of conjugation of the linker to the DT4-yl and amine groups may be selected, in order to optimise accessability, stability in circulation, activity in the target tissue, etc.

According to a second aspect of the invention, there is provided a novel compound consisting of insulin or a functional equivalent thereof having covalently bound to the alpha-amine group of the B1 residue an N—C₁₋₄-alkanoyl-(di-, tri- or tetra-) iodothyronyl group.

In this aspect of the invention, again the thyronyl group may be conjugated to the B1 residue through a linker. The linker may be as described above.

In this aspect of the invention the thyronyl group is preferably a 3,3′,5,5′-tetraiodothyronyl group, preferably DT4.

The C₁₋₄-alkanoyl group on the thyronyl amine group is preferably acetyl, or may alternatively be propanoyl.

According to a third aspect of the invention there is provided a novel compound consisting of insulin or a functional equivalent thereof having covalently bound thereto a thyroid hormone, by a linker which has the general formula —OC—(CR₂)ₙ—NR¹—, in which the —OC is joined to the insulin, the NR¹— is joined to the thyroid hormone, each R is independently selected from H and C₁₋₄-alkyl, n is an integer of at least 11 and R¹ is H, C₁₋₄-alkyl or C₁₋₄-alkanoyl.

In this third aspect of the invention the —OC group of the linker is joined to the alpha amine group of the B1 residue of insulin, or functional equivalent of insulin. Alternatively, the linker may be joined to another free amine group on the insulin molecule, such as the epsilon-amino group of the B29 lysine residue. The conjugation with insulin should leave the active sites of insulin available for the insulin to have its endogenous metabolic effect.

In this third aspect of the invention, the thyroid hormone is preferably LT4 or DT4.

Preferably the linker is —OC—(CH₂)₁₁—NH—.

According to a fourth aspect of the invention there is provided a new method in which the novel N-alkanoated derivatives or other N-alkanoylated compounds may be formed, comprising the steps:

a) reacting i) a thyronyl reagent of the general formula I

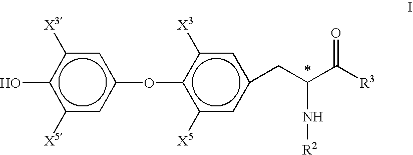

in which each group X³, X^{3′}, X⁵ and X^{5′} is selected from H and I; provided that at least two of the groups represent I;

R² is an amine protecting group; and

R³ is a carboxylic activating group,

with ii) an amine compound ₘ(R⁴N)R⁵(NH₂)ₚ

in which R⁵ is a (m+p)-functional organic group;

R⁴ is an amine protecting group other than R²;

m is 0 or an integer of up to 10; and

p is an integer of at least 1,

to produce a protected intermediate

b) the protected intermediate is treated in a selective amine deprotection step under conditions such that protecting group R² is removed, but any R⁴ groups are not removed to produce a deprotected intermediate; and

c) the deprotected amine group of the deprotected intermediate is acylated by a C₁₋₄ alkanoyl group in an alkanoylation step to produce an N-alkanoylated compound.

In this aspect of the invention the amine compound may be insulin or a functional equivalent thereof. The above process may be applied to oligo- or poly-peptide actives other than insulin, which have a free amine group for acylation by the thyronyl reagent. Preferably the technique is applied to insulin, most preferably the alpha-amino group of the B1 residue of insulin.

The protecting groups R² and R⁴ are selected so as to allow selective deprotection in step b of the process. Preferably R² is a Boc group (tertiary-butoxycarbonyl). Deprotection is preferably carried out using conventional deprotection methodology, either using hydrochloric acid/acetic acid mixtures or, preferably, using trifluoroacetic acid.

The R⁴ protecting group is selected such that it is not removed by the selective deprotection step b. Conveniently it is a Msc group (methylsulphonylethoxy carbonyl). Such groups may be removed under conditions which do not result in cleavage of the bond formed in step a, nor of the bond formed in the alkanoylation step. Suitable conditions for a subsequent non-selective deprotection step are alkaline, for instance using sodium hydroxide.

The novel process minimises racemisation of the asymmetric carbon atom (C*)of the thyronyl group. Suitably the asymmetric carbon atom is in the L configuration, although the D-stereoisomer may be used.

The inventions are illustrated in the accompanying examples.

Abbreviations:

Msc=methylsulphonylethyloxycarbonyl

Boc=tert. butyloxycarbonyl

DMF=dimethylformamide

DMSO=dimethylsulfoxide

mp=melting point

ONSu=N-oxysuccinimide ester

TFA=trifluroacetic acid

NMM=N-methylmorpholine

DCC=dicyclohexylcarbodimide

NHS=N-hydroxylsuccinimide

### EXAMPLES

### Reference Example 1

Msc-L-thyroxine (1)

776 mg (1 mmol) L-thyroxine in 2 ml dimethylsulfoxide was reacted with 530 mg (2 mmol) Msc-ONSu in the presence of 139 μl (1 mmol) triethylamine at room temperature for 18 hours. Then the solution was pipetted into 20 ml ice cold HCl solution (pH2). The precipitate was isolated by centrifugation washed three times with an aqueous HCl solution and dried in vacuo.

Yield: 843 mg (91% of theory),

RP-KPLC purity: 99.1%

### Reference Example 2

The synthesis was carried out analogous to that of (1) using D-thyroxine as starting material.

Yield: 819 mg (88% of theory)

RP-HPLC purity: 98.4%

### Reference Example 3

Boc-L-thyroxine (3)

776.0 mg (1 mmol) L-thyroxine was dissolved in 5 ml dimethylsulfoxide. The pH of the solution was adjusted to 9 by adding Na₂CO₃. After cooling the solution to 0° C. 275.0 mg (1.2 mmol) di-tert.-butyl-dicarbonate (solid) was added under stirring. After stirring for 4 hours at 0° C. the solution was pipetted into a an ice-cold aqueous HCl solution (pH 2). After centrifugation the precipitate was washed twice with and aqueous HCl solution and dried in vacuo.

Yield: 721 mg (82% of theory)

RP-HPLC purity: 98.4%

### Reference Example 4

N-Boc-12-aminolauric acid (4) (N-Boc-12-aminododecanoic acid)

A solution of 2.74 g (12.7 mmol) 12-aminolauric acid in 45 ml 1,4-dioxane/water (2/1; v/v) was cooled to 0° C. and adjusted to pH 9 with 1N NaOH. After addition of 4.80 g (22.0 mmol) di-tert.-butyl-dicarbonate, dissolved in 10 ml 1,4-dioxane, the solution was stirred for 4 hours, maintaining a constant pH of 9 by adding 1N NaOH if necessary. The organic solvent was evaporated in vacuo. The aqueous part was adjusted to pH 2 with a 10% aqueous KHSO₄ solution and was extracted tree times with acetic acid ethyl ester. The joined organic phases were washed once with 10 ml of a cold saturated NaCl solution, twice with water, dried, filtered, and concentrated until precipitation began. After keeping for 18 hours at +4° C. the product was isolated by filtration and dried in vacuo.

Yield: 3.7 g (92% of theory)

### Reference Example 5

N-Msc-D-thyroxine-N-oxysuccinimide ester (5)

To a solution of 200.0 mg (0.22 mmol) of (2) and 25.3 mg (0.22 mmol) N-hydroxysuccinimide in 2 ml THF 45.3 mg (0.22 mmol) N,N′-dicyclohexylcarbodiimide in 0.42 ml THF were added under stirring at 0° C. After 3 hours dicyclohexyl urea was removed by filtration. The solution was concentrated and kept for 18 hours at +4° C. The product was isolated by filtration and dried in vacuo.

Yield: 176 mg (79% of theory)

RP-HPLC purity: 76.6%

### Reference Example 6

N-Boc-L-thyroxine-N-oxysuccinimidylester (6)

The synthesis was carried out analogous to that of (5) using (3) as starting material.

Yield: 1912 mg (87% of theory)

RP-HPLC purity: 82.8%

### Example 1

B1-D-thyroxyl-insulin (Human) (7)

To a solution of 100.0 mg (approx. 0.016 mmol) A1,B29-Msc₂-insulin (prepared according to Schüttler and Brandenburg, Hoppe-Seyler's Z. Physiol. Chem. 360, 1721-1725(1979)) and 18.0 μl (0.16 mmol) N-methyl-L-morpholine (NMM) in 2 ml DMF 93.1 mg of 2 in 0.2 ml DMF were added. After stirring for 6 hours at room temperature the insulin derivative was precipitated with ether, isolated by centrifugation, washed tree times with ether and dried in vacuo. Msc groups were removed by treatment with NaOH/dioxane/water at 0° C. and 17 was first purified by gel filtration on Sephadex G-50 fine as described (Geiger et al, Chem. Ber. 108,2758-2763 (1975)), lyophilized, and then purified by RP-HPLC.

Yield: 34.9 mg (33.3% of theory)

RP-HPLC purity: 99.6%

### Reference Example 7

B1-L-thyroxyl-insulin (Human) (7a)

The synthesis, carried out in an analogous way to that of (7) from 100.0 mg A1, B29-Msc₂-insulin and (1), gave 74 mg (70.4% of theory) (7a) in a purity of 88.9% after removal of Msc groups, and 37.2 mg (35.4% of theory) after RP-HPLC purification. RP-HPLC purity was 99.8%.

### Example 2

2.1 Synthesis of B1-(T4-Aminolauroyl)-insulin (Human)

For 12-aminolauric acid n=11 First, A1,B29-Msc₂-insulin was reacted with 6 equivalents of (4), which had been pre-activated with dicyclohexylcarbodiimide/hydroxybenzotriazole (DCC/HOBt) (König & Geiger, Chem. Ber. 103, 788-798) for 1 h at 0° C. and 1 h at room temperature. After 70 min at room temperature the reaction was complete, and the protein was precipitated. Subsequently, the Boc groups were selectively removed with TFA.

The intermediate (B1-(12-aminododecanoyl)-A1,B29-Msc₂-insulin was isolated in a yield of 80% and a purity of 59%.

2.2 B1-L-thyroxyl-(12-aminolauryl)-insulin (Human) (8)

To a solution of 103.4 mg B1-aminolauroyl-A1,B29-Msc₂-insulin and 18.0 μl N-methyl-L-morpholine in 2 ml DMF 134 mg of I in 0.2 ml DMF were added. After stirring for 6 hours at room temperature the insulin derivative was precipitated with ether, isolated by centrifugation, washed with ether and dried in vacuo. The protecting groups were removed by treatment with NaOH/dioxane/water at 0° C. VIII was purified by first by gel filtration on Sephadex G-50 fine and subsequently by semi-preparative RP-HPLC.

Yield: 29.5 mg (27.3% of theory)

RP-HPLC purity: 97.6%

2.3 B1-[D-thyroxyl-(12-aminolauryl)]-insulin (Human) (9)

The synthesis was carried out analogous to that of (8) using (5) as the starting material.

Yield: 26.7 mg (25% of theory)

RP-HPLC purity: 98%

### Example 3

Two analogues with modified thyroid moiety, in which the α-amino group was acetylated, have been synthesized and characterized.

Acetylation of LT4 was quantitative in acetic acid anhydride at 40° C. N-Acetyl-LT4 was activated with DCC/NHS and directly coupled to a) partially protected insulin (A1, B29 (MSc)₂ insulin) and to b) B1-12-aminododecanoyl (Msc)₂-insulin, following the procedure described.

However, after deblocking RP-HPLC revealed an apparent non-homogeneity of the product.

MS analysis of the separated individual peaks as well of the mixture gave in all cases the mass of 6609 calculated for B1-N-acetyl-L-T4-insulin. We believe this indicates racemisation annuity the synthesis

### Example 4

In order to avoid the racemisation uncovered in example 3, a stereo-conservative synthesis of B1-N-acetyl-LT4-insulin via an orthogonal protecting group tactic was designed.

4.1 N-acetyl-L-thyroxyl]-insulin (Human) (10)

To a solution of 100.0 mg A1,B29-Msc₂-insulin and 18.0 μl N-methyl-L-morpholine in 2 ml DMF 134 mg of 3 in 0.2 ml DMF were added. After stirring for 6 hours at room temperature the insulin derivative was precipitated with ice cooled ether, isolated by centrifugation, washed tree times with ether and finally dried in vacuo. The Boc group was removed by treatment with TFA followed by purification via gel filtration on Sephadex G-50 fine and lyophilization. In order to acetylate the amino function of the thyroxyl moiety 50.0 mg of B1-L-thyroxyl-A1,B29-Msc₂-insulin were dissolved in one ml DMF and reacted with 22.9 mg acetic acid succinimide ester for 2 hours at room temperature. The protein was isolated by precipitation in ice cooled ether. The final removal of the Msc groups was carried out in NaOH as described.

Final purification was by semi-preparative RP-HPLC.

Yield: 38.3 mg (36% of theory)

RP-HPLC purity: 99.4%

4.2 B1-((N-acetyl-L-thyroxyl)-(12-aminolauryl))-insulin (Human) (11)

The synthesis followed the procedure described for 10, using B1-aminolauroyl-A1,B29-Msc₂-insulin as intermediate. First, Boc-LT4 was coupled. After cleavage of the Boc group, selective acetylation with acetic acid succinimide ester was performed. Basic removal of Msc groups and semipreparative RP-HPLC gave 11.

Yield: 23.5 mg (21% of theory)

RP-HPLC purity: 98.2%

MALDI-TOF-MS was applied to determine the molecular masses of the Thyroid-Insulin-conjugates. During the measurements, partial de-iodination of the thyroid moiety was observed with all conjugates. In table 1 the masses found and calculated are compiled for the spectra masses.

TABLE 1
Molecular masses of the Thyroid-Insulin-Conjugates

| | [MH]⁺ | [MH]⁺ |
|---|---|---|
| Analogue | (calc.) | (found) |
| B1-LT4-Insulin (reference) | 6567 | 6567 |
| B1-DT4-Insulin | 6567 | 6566 |
| B1-N-Acetyl-LT4-insulin | 6609 | 6609 |
| B1-LT4-(12-Aminododecanoyl)- | 6765 | 6762 |
| insulin | | |
| B1-DT4-(12-Aminododecanoyl)- | 6765 | 6765 |
| insulin | | |
| B1-N-Acetyl-L-T4-(12-amino- | 6807 | 6806 |
| dodecanoyl)insulin | | |

### Example 6

Binding Properties of Thyroid-Insulin-Conjugates to Insulin Receptor

The thyroid-insulin conjugates combine in one molecule thyroid- as well as insulin-specific properties.

As the insulin-specific property, binding to insulin receptors in vitro was studied. Receptor binding was determined in competition assays with {Tyr-(¹²⁵I)^{A14}}-Insulin in cultured IM-9 Lymphocytes. Because of the designed affinity of the substituted insulin conjugates towards serum albumin the standard 1% solution of BSA was replaced by 1% γ-globulin (suppression of non-specific binding).

Relative binding was calculated using the program Prism via non-linear curve-fitting.

The receptor affinities are compiled in Table 2.

TABLE 2
Relative binding affinities of Thyroid-Insulin-conjugates to insulin
receptor

| Analogues | rel. Binding affinities in % |
|---|---|
| B1-LT4-Insulin (reference) | 498 |
| B1-DT4-Insulin | 12.3 |
| B1-N-Acetyl-LT4-insulin | 30.0 |
| B1-LT4-(12-Aminododecanoyl)insulin | 3.9 |
| B1-DT4-(12-Aminododecanoyl) | 7.3 |
| insulin | |
| B1-N-Acetyl-LT4-(12- | 1.4 |
| Aminododecanoyl)insulin | |

Replacing LT4 by the stereo isomeric DT4 brings about as marked reduction in the affinity from about 50 to 12.3%. Acetylation of the amino group of L-thyroxine reduces the C₁₂ affinity to 30%. Introducing the spacer arm leads to pronounced loss of affinity in all three cases.

### Example 7

Binding Studies to the Plasma Protein TBG

The optical bio-sensor IAsys makes it possible to record biomolecular interactions in real time and thus kinetical studies. We studied the binding of the Thyroid-Insulin conjugates B1-LT4-Insulin (reference), B1-DT4-Insulin and B1-N-acetyl-LT4-insulin to the plasma protein thyroxine binding globulin(TBG).

The surface of the cuvette is covered with a carboxymethylated dextran matrix (CMD), to which the plasma protein TBG is immobilized.

Immobilization of TBG to the carboxymethylated matrix is detected via the change of the resonance angle.

For the kinetical studies the Thyroid-Insulin conjugates were injected into the microcuvette in dilution series of 200, 300, 400 and 500 μg/ml in HBS/Tween-buffer at 25° C. To test for reproducibility, all measurements were repeated 3 times.

As a control, native insulin was injected at high concentration (500 μg/ml). While injection leads to a buffer-jump, association cannot be observed. Removal of the insulin solution and injection of blank buffer caused another buffer-jump, but there was no sign of dissociation. Thus, non-specific binding of insulin to the immobilized plasma protein can be excluded. For further measurements the surface of the microcuvette was rinsed several times with buffer.

Determination of “on-rate” constants kₒₙ at various ligand concentrations c_{L} allow kₒₙ to be plotted against c_{L} according to equation (4). This gives the association rate constant k_{A} from the slope and the dissociation rate constant k_{D} at C₂=O. It has, however, to be taken into account that the error of k_{D} becomes too large when k_{D}<0,01 s⁻¹ (IAsys, METHODS GUIDE).

kₒₙ=k_{D}+k_{A}·c_{L} 4

In the binding studies with Thyroid-Insulin-conjugates to immobilized TBG the good reproducibility of the individual determinations has to be noted.

The association and dissociation curves of the 3 Thyroid-Insulin-conjugates indicated in Table 3 were analyzed with the program Fast-fit. For quantification of association single-phasic curve-fitting was chosen, since the values for two-phase fitting showed larger fluctuations.

The association rate constants for the conjugates are listed in Table 3.

TABLE 3
Association constants of the Thyroid-Insulin Conjugates to the
plasma protein TBG.

| Analogues | k_{A}/(10⁵ M⁻¹s⁻¹) |
|---|---|
| B1-LT4-Insulin (ref) | 3.23 ± 0.89 |
| B1-DT4-Insulin | 1.21 ± 0.39 |
| B1-N-Acetyl-LT4-insulin | 0.5′* |

*no determination with the program Fast-fit possible Estimated 0.5

k_{A} for B1-LT4-Insulin was markedly larger than k_{A} for B1-DT4-Insulin. Plotting of kₒₙ-values of B1-N-acetyl-LT4-insulin against ligand concentration gave a large dispersion, and quantitative evaluation was not possible. The individual curves resembled, however, very much those of B1-DT4-Insulins.

Evaluation of dissociation was also via single phase curve-fitting, for the same reasons as above. The dissociation constants k_{D} of the Thyroid-Insulin-Conjugates under study are compiled in Table 4.

TABLE 4
Dissociation constants of the Thyroid-insulin conjugates to the
plasma protein TBG.

| Analogues | k_{D}/(10⁻² s⁻¹) |
|---|---|
| B1-LT4-Insulin (ref) | 5.56 ± 2.39 |
| B1-DT4-Insulin | —* |
| B1-N-Acetyl-LT4-insulin | 4.49 ± 0.70 |

*no determination with the program Fast-fit possible

k_{D} of B1-LT4-Insulin was about 20% larger than k_{D} of B1-N-acetyl-LT4-insulin. Inspite of good reproducibility within the various concentrations, the fluctuations observed did not allow calculation of k_{D} for B1-DT4-Insulin.

### Example 8

Structural Characteristics of Thyroid-Insulin-Conjugates

The analogues B1-LT4-Insulin and B1-LT4(12-aminododecanoyl)insulin have been analyzed by CD-spectroscopy.

B1-LT4-Insulin was studied at concentrations 0,017; 0,17 and 0,88 g/l, as well as at 0,88 g/l in the presence of 0.4 equivalents of zinc ions. Under all conditions, the same spectrum was recorded. Neither increase of concentration nor the presence of zinc led to changes in ellipticity. The insulin-typical maximum at 195 nm was always seen.

In the near UV the concentration-dependency of the ellipticity is only small. In contrast to native insulin, there was a positive band at 252 nm, which, however, sank upon addition of zinc to a level common for insulin. At 275 nm, a profile typical for insulin was observed. However, the spectrum did not reach the value typical for 2Zn-hexamers (=−305 grad·cm²×dmol⁻¹).

With native insulin, addition of phenol induces the T→R transition, where the extended N-terminus of the B-chain is transformed into an α-helical structure. In the near UV, this is accompanied by an increase of negative ellipticity at 251 nm to a value of approx. 400. In the case of B1-LT4-Insulin, again there is only a small hint in this direction.

B1-LT4-(12-aminododecanoyl)insulin was analyzed in the far UV at concentrations 0,02; 0,20 and 0,68 g/l. In addition, the determination at 0,68 g/l was performed in the presence of 0,33 equivalents of zinc. B1-LT4(12-aminododecanoyl)insulin exhibited an insulin-typical maximum at 195 nm. Increase of concentration and addition of zinc left the spectrum unchanged.

In the near UV, the hybrid B1-LT4-(12-aminododecanoyl)insulin was studied at concentrations of 0,02 and 0,68 g/l (FIG. 37). In contrast to B1-LT4-Insulin, B1-LT4-(12-aminododecanoyl)insulin showed no positive band at 255 nm. At 275 nm th4 spectrum resembled that of insulin. The ellipticity sank below −200, but did not reach the value for insulin (−305).

### Example 9

Binding Studies to Liver Plasma Membrane

9.1 Isolation of Rat Liver Plasma Membrane (LPM)

Rat liver plasma membrane (LPM) was isolated to be used in equilibrium binding assays as the source of insulin receptors. LPM actually contains not only plasma membrane, but also membrane of the nucleus, mitochondria, Golgi bodies, endoplasmic reticulum and lysosomes. When cell membranes are fragmented, they reseal to form small, closed vesicles—microsomes. Therefore, LPM can be separated into a nuclear and a microsomal component. Each component can be separated into a light and a heavy fraction, which in turn, can be separated into further subfractions. Plasma membranes, where insulin receptors reside, are found in the light fractions, but the present aim was to obtain the microsomal light fraction only, since the nuclear light fraction usually produces variable results in the binding assay. The method first described by Neville (1960) was used to isolate plasma membrane fractions from fresh rat livers.

9.2.1 Fast Protein Liquid Chromatography (FPLC)

To ascertain the binding of the insulin or the analogue to the thyroid hormone binding proteins (THBPs), they were incubated overnight at 4° C. The bound and unbound species were separated by molecular weight with FPLC. As shown in Table 1, the binding of H-Ins, LT₄-Ins, DT4-Ins and LT4-(CH₂)₁₂-Ins (synthesised according to Example 2) to normal human serum, HSA (human serum albumin) and TBG (thyroxine binding globulin) were studied.

The THBP concentrations used were physiological, except TBG, due to reasons of costs.

TABLE 1
The binding of each analogue to each THBP were studied

| | Thyroid hormone | | Physiological |
|---|---|---|---|
| Insulin or | binding | Concentration | THBP |
| Insulin Analogues | proteins (THBPs) | of THBPs used | concentration |
| H-Ins | TBG | 0.238 μM | 0.27 μM |
| DT4-Ins | HSA | 5% (w/v), | 4.24% (w/v), |
| | | or 757 μM | or 640 μM |
| LT₄-Ins | Normal human | | |
| | serum | | |
| LT4-(CH₂)₁₂-Ins | (TBG, albumin, | | |
| | prealbumin) | | |

HSA = human serum albumin
TBG = thyroxine binding globulin

9.2.2 Dilution of THBPs and Incubation with Analogues

Solutions (0.5 ml) of THBPs were prepared in FPLC buffer as follows and then vortexed:

Normal human serum—used undiluted.

HSA (5% w/v)—diluted 1:4 from HSA (20% w/v).

TBG—10 μl of stock TBG (0.1 mg/0.13 ml) was added to 0.5 ml buffer. The amount of HSA in the FPLC/Barbitone/HSA buffer (0.2%) was too small to significantly alter the binding of TBG to the analogues.

H-Ins (100 μl of 0.276 μM) or analogues was added to the THBP solution. It was vortexed and incubated at 4° C. for ≈16 hours or overnight. Before FPLC, it was vortexed again, and filtered through a syringe filter of pore size 0.2 μm (Acrodisc® LC13 PVDF from Gelman, UK) to remove bacteria and serum precipitates.

9.2.3 Fractions Are Collected from the Column

The fraction tubes (LP3 tubes) were coated with 50 μl 3% (w/v) HSA to prevent the analogues from adsorbing to the tubes' inner surfaces. The fraction size was programmed as 0.50 ml. Immunoreactive insulin in each fraction was assayed with radioimmunoassay on the same day.

9.2.4 Radioimmunoassay (RIA) for Insulin

A double-antibody radioimmunoassay (RIA) was performed to determine the concentrations of H-Ins or insulin analogue in each FPLC fraction, using insulin-specific antibodies.

The assay was calibrated using insulin standards. Before the insulin standards and FPLC fractions can be assayed, their HSA concentrations were standardized, by diluting them with Barbitone/HSA(0.2% w/v) buffer and FPLC/Barbitone/HSA buffer. A double dispenser (Dilutrend, Boehringer Corporation London Ltd) was used to add the appropriate volume of buffer and standard or FPLC fractions into the labelled LP3 tubes. The total volume of each tube was 500 μl. In addition, three tubes of NSB (non-specific binding), containing the standardized HSA concentration, were prepared with, Barbitone/HSA(0.2% w/v) and FPLC/Barbitone/HSA buffers. Table 2 summarizes the dilution of the standards and FPLC fractions, as well as the preparation of the TC and NSB tubes.

TABLE 2
Contents of the final assay tubes

| | Final Assay Tubes | | | |
|---|---|---|---|---|
| | | | | FPLC |
| Contents | TC | NSB | Standard | fractions |
| Std. Solutions | — | — | 50 | — |
| FPLC/BARBITONE/HSA | — | 350 | 350 | — |
| buffer | | | | |
| Barbitone/HSA(0/2%) Buffer | — | 150 | 100 | 150 |
| FPLC sample | — | — | — | 350 |
| [¹²⁵l]insulin tracer | 100 | 100 | 100 | 100 |
| Primary Ab | — | 0 | 100 | 100 |
| Secondary Ab | — | 100 | 100 | 100 |
| Total volume | 100 | 800 | 800 | 800 |

All volumes in μl.

*Replace with 100 μl Barbitone/HSA(0.2% w/v) buffer

Std.=standard; Ab=antibody.

9.2.5 Addition of [¹²⁵I]Insulin Tracer

An aliquot of [¹²⁵I] insulin tracer was added to Barbitone/HSA (0.2% w/v) buffer of an adequate volume (100 μl per tube). The radioactivity in 100 μl of the resulting tracer solution was counted in the γ-counter, and the counts per minute (cpm) should lie between 3000-5000 cpm. ANSA (2 mg/ml) was dissolved in the solution, and it functioned to displace the T₄ moieties on the analogues from the THBPs, since the THBP could be shielding the insulin moiety that was to be assayed. Finally, 100 μl of this solution was added to every tube.

9.2.6 Addition of Primary Antibody (W12) and Incubation

The primary antibody, W12, is a polyclonal, guinea-pig anti-insulin antibody. It recognises epitopes away from the B1 residue of the insulin molecule, so that the T₄ moiety, which is linked to the B1 residue, will not hinder the binding W12. It was diluted to 1:45,000 in Barbitone/HSA(0.2% w/v) buffer, and 100 μl was added to every tube, except the TC and NSB tubes. Finally the tubes were vortexed in a multi-vortexer (Model 2601, Scientific Manufacturing Industries, USA) and incubated at room temperature for about 16 hours.

9.2.7 Addition of Secondary Antibody (Sac-Cel)

The secondary antibody, Sac-Cel (IDS Ltd., AA-SAC3), is a pH7.4, solid-phase suspension that contains antibody-coated cellulose. It was diluted 1:1 (v/v) with Barbitone/HSA(0.2% w/v), and 100 μl was added to all tubes (except TC), vortexed, and incubated at room temperature for 10 min. 1 ml distilled water was added to the tubes prior to centrifugation to dilute the solution, thereby minimising non-specific binding.

9.2.8 Separation of Free and Bound Species

To separate the free and antibody-bound species, the tubes were centrifuged at 2,500 rpm to 20 min in a refrigerated centrifuge (IEC DPR-6000 Centrifuge, Life Sciences International) set at 4° C. The tubes were then loaded into decanting racks. The supernatant, containing the free species, were decanted, by inverting the trays quickly over a collection tub. Care was taken to prevent the pellet from slipping out, and the tubes were wiped dry to remove the traces of supernatant. The combined supernatant was later disposed according to the laboratory's safety guidelines in the sluice. Finally, the samples, together with the TC and NSB tubes, were counted in the γ-counter using a programme for RIA(RiaCalc).

9.3 Equilibrium Binding Assay

This equilibrium binding assay determines the analogues affinity to the insulin receptors on the LPM, both in the presence and absence of the THBPs. In brief, a fixed amount of [¹²⁵I]insulin tracer was incubated with the analogue at different concentrations, together with a fixed volume of LPM, such that the analogue inhibited the tracer from binding to the insulin receptors. The amount of bound tracer was counted in the γ counter after separating the bound and free species by centrifugation. The results were used to calculate the ED50 (half effective dose) and binding potency estimates relative to H-Ins, or, in assays investigating the effects of added THBPs, relative to the analogue in the absence of THBPs.

9.4 Results

9.4.1 Radioimmunoassay (RIA)

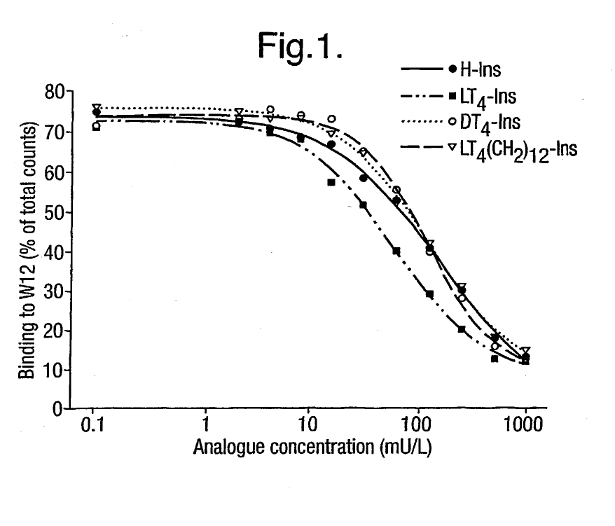

Double antibody RIA was used to quantify the immunoreactive insulin (IRI) in the FPLC fractions. The validity of using RIA to quantify the novel analogues, whose antibody binding behaviour was unknown, was confirmed by assaying standard solutions of H-Ins, DT4-Ins LT₄-Ins and LT4 (CH₂)₁₂-Ins FIG. 1 shows the inhibition of [¹²⁵I] insulin binding to the primary antibody W12 by H-Ins and the analogues. Their ED50s were 1065 pM (H-Ins), and 417.3 pM (LT₄-Ins), 818.3 pM (DT4-ins) and 855.9 pM (LT4-(CH₂)₁₂-Ins). Since ED50's for H-Ins, DT4-Ins and LT4-(CH₂)₁₂-Ins appeared similar (no statistical analysis was done due to small size), it can be assumed there are no major differences in the antibody recognition of the insulin moiety on the novel analogues as compared to H-Ins.. The standard curve for LT₄-Ins, however, was shifted to the left of the other curves, which could signify a lower binding to W12.

9.4.2 Fast Protein Liquid Chromatography (FPLC)

FPLC was used to study the binding of the insulin and the analogue to the THBPs (normal human serum, HSA 5% w/v, TBG 0.238 μM). IRI content in each fraction was assayed by RIA.

a) Non-Specific Binding of THBPs

Non-specific binding of the THBPs to the antibodies in RIA was measured by eluting the THBPs alone, and the fractions were assayed for IRI. They all showed negligible amounts of IRI.

b) Elution Profiles

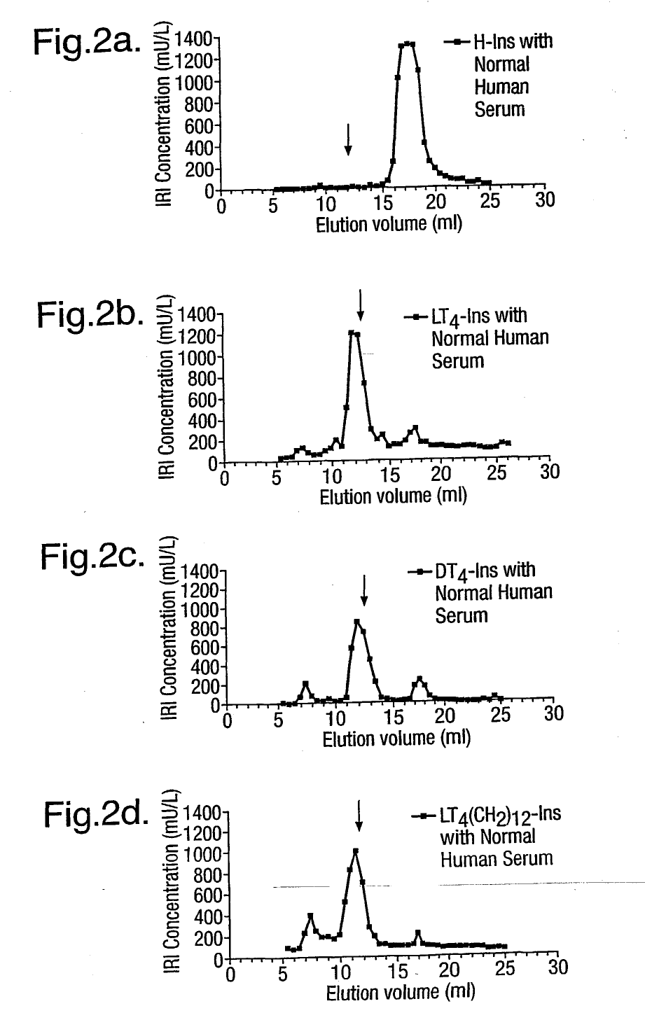

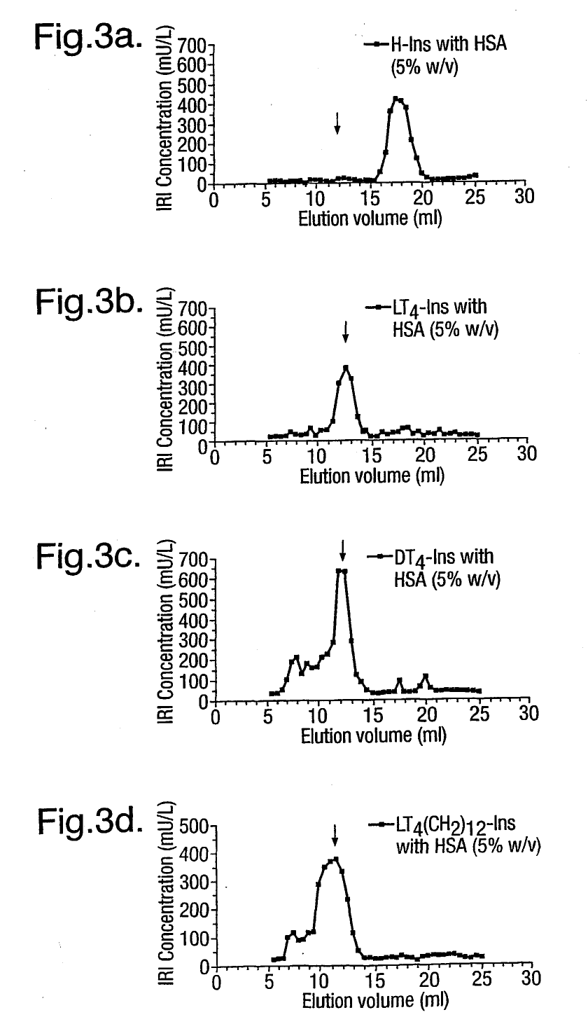

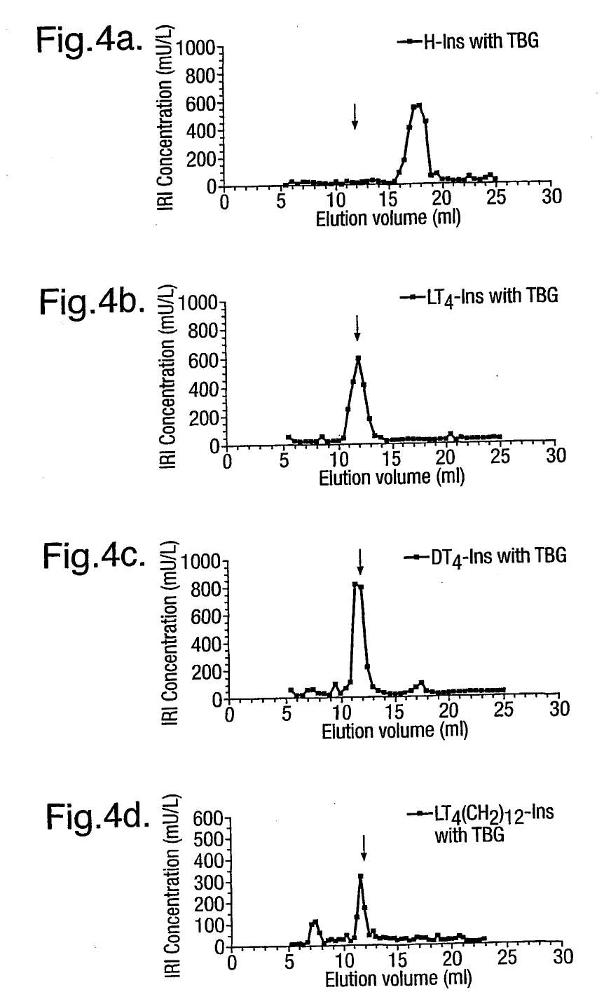

FIGS. 2a-d show the elution profiles of H-Ins, LT₄-Ins, DT4-Ins and LT4-(CH₂)₁₂-Ins, respectively after overnight incubation with the normal human serum. FIGS. 3a-d show the elution profiles of the conjugates after overnight incubation with 5% human serum albumin (HSA). FIGS. 4a-d show the elution profiles of H-Ins and LT₄-Ins, respectively, after overnight incubation with 0.238 μM TBG. The calculated % bound and % free values are included in Table 3. Appearances of the THBPS, as detected by UV absorbance on the original chromatogram (which was not sensitive enough to detect the analogues) are also indicated as arrows on the elution profiles. The shadowed box represents the bound fractions; the clear box represents free fractions.

H-Ins

The calculated % bound for H-Ins to each THBP was significantly lower than the % bound of the LT₄-Ins analogues to the same THBPs (p<0.05). Nevertheless, the % of bound H-Ins was not completely negligible. Background binding of 9.02% to HSA and 9.85% to TBG was observed (FIGS. 3A, 4A).

LT₄-Ins, DT₄-Ins and LT₄(CH₂)₁₂-Ins

The thyroxyl-linked analogues all showed substantial binding (>60%) to the THBPs (Table 1). For normal human serum, teh % bound to DT₄-Ins were both significantly higher than that to LT₄-Ins (p<0.05). For HSA (5% w/v), the % bound to LT₄(C₂)₁₂-Ins was significantly higher than that to both LT₄-Ins (p, 0.05). For TBG (0.238 μM), the % bound to DT₄-Ins was significantly higher than that to both LT₄-Ins and LT₄(CH₂)₁₂-Ins (p<0.05).

9.4.3 Equilibrium Binding Assays

Equilibrium binding assays to insulin receptors on LPM were performed for H-Ins LT₄-Ins DT4-Ins and LT4(CH₂)₁₂-Ins. In addition, the effects of added THBPs (normal human serum 45%, HSA 5% w/v, TBG 0.13 μM) on the two novel analogues were also studied.

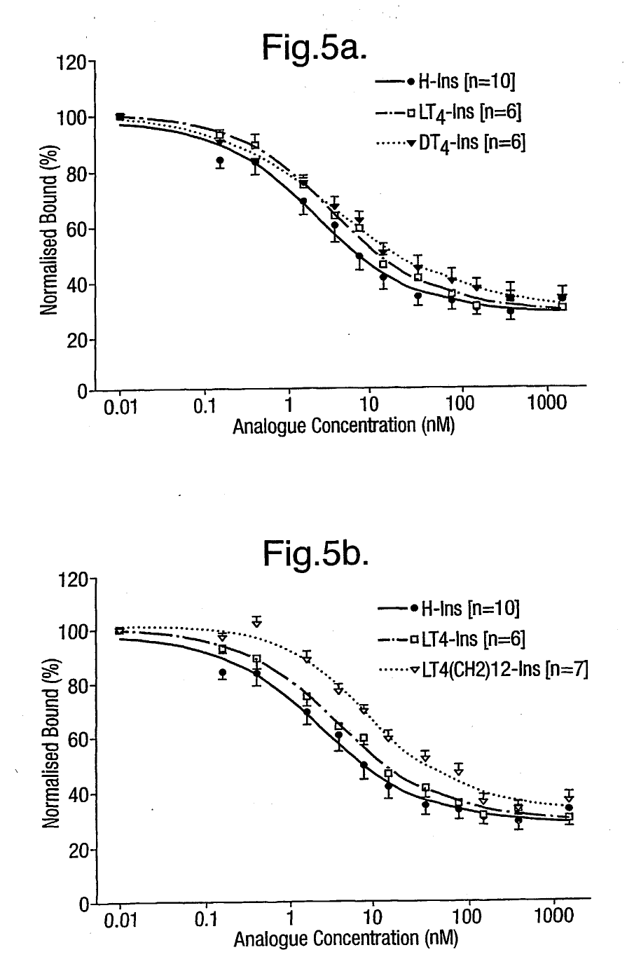

Equilibrium binding curves, which represent the inhibition of [¹²⁵I] insulin binding to LPM by H-Ins and the analogues, are shown in FIGS. 5a and b and 6 to 11. Each curve represents the mean results of several assays, and the mean ED50s of the assays are shown in Table 4.

Relative potency estimates (RPE) of the analogues are summarized in Table 5. The values showed insignificant heteroscedasticity (Barlett X² test, p<0.05), but some showed significant non-parallelism (F<0.05).

Binding in the Absence of THBPs

FIGS. 5a and 5b show the inhibition of ¹²⁵I-insulin binding to LPM by H-Ins and the conjugates.

The binding curve of LT₄-Ins, DT₄-Ins and LT₄(CH₂)₁₂-Ins were all shifted to the right of the H-Ins curve (FIGS. 5a and b and their ED50s were all significantly higher than H-Ins' (p<0.05). The ED50s of two novel analogues, DT₄-Ins LT₄(CH₂)₁₂-Ins, were both higher than LT₄-Ins' (p<0.05), but were not significantly difference from each others'.

The RPE of the three analogues relative to H-Ins were all 100%. LT₄-Ins was 63.5% (40.5-96.7%), DT₄-Ins was 45.4% (27.9-70.0%), and LT₄(CH₂)₁₂-Ins was the least potent at 22.6% (14.1-33.8%).

Binding in the Presence of THBPs

For the binding assays performed in the presence THBP, shifts in the binding curves and the changes in ED50s and RPE are described relative to binging of the same analogue in the absence of THBP.

Normal Human Serum (45% v/v)

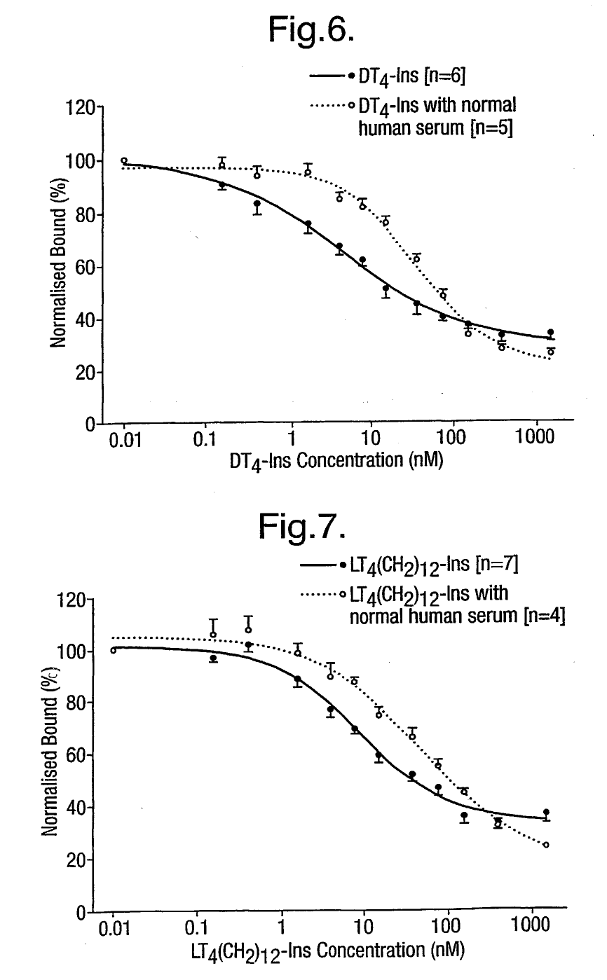

FIG. 6 shows the inhibition of ¹²⁵I-Ins binding to LPM by DT4-Ins in the presence and absence of normal human serum. FIG. 7 shows the coresponding curves for LT4(CH₂)₁₂Ins.

When normal human serum (45% v/v) was added (FIGS. 6, 7), the binding curves of DT₄-Ins and LT₄(CH₂)₁₂-ins was significantly higher than binding in the absence of THBP (p<0.05), and its RPE was only 21.0% (11.3-34.5%). For DT₄-Ins, however, the slope of the linear portion of the binding curve was significantly greater, such that the shift was non-parallel. Its ED50 and RPE, therefore, cannot be validity compared to its binding without THBP. It was also of interest that there was no displacement of {¹²⁵I] insulin up till ≈5 nM, and there was cross-over of the two curves at ≈110 nM.

HSA (5% w/v)

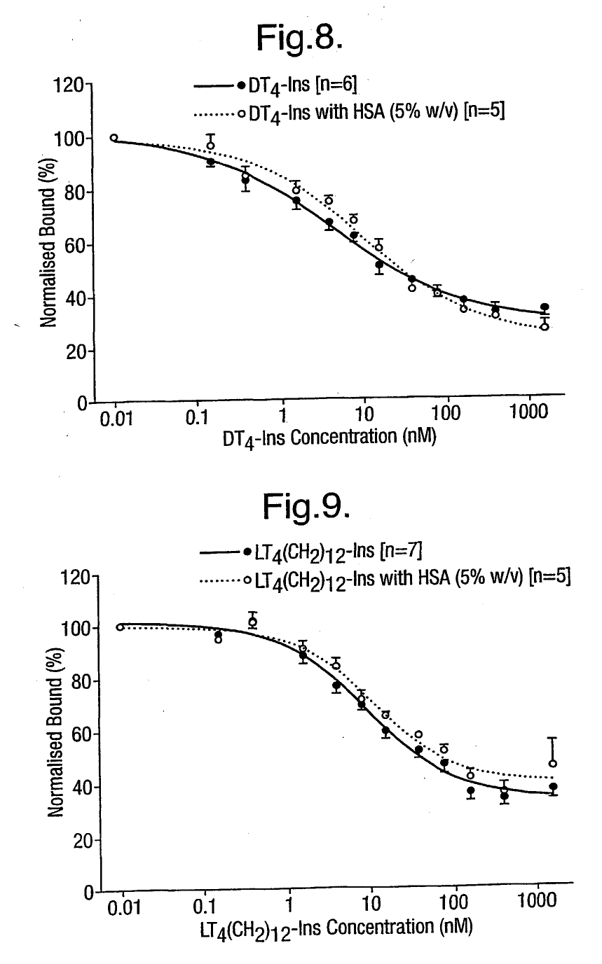

FIG. 8 shows the inhibition of ¹²⁵I-Ins binding to LPM by DT4-Ins in the absence and presence of 5% HSA. FIG. 9 shows that corresponding curves for LT4(CH₂)₁₂Ins.

In the presence of HSA (5% w/v), the binding curves of both DT₄-Ins and LT₄(CH₂)₁₂-ins were shifted to the right, but only the ED50 of LT₄(CH₂)₁₂-Ins was significantly higher than binding in the absence of THBP (p<0.05). The RPE for DT₄-Ins with HSA is 67.3% (37.8115.0%) and the RPE for LT₄(CH₂)₁₂-Ins with HSA is 92.8% (66.6-129.2%).

TBG (0.135 μM, 0.27 μM)

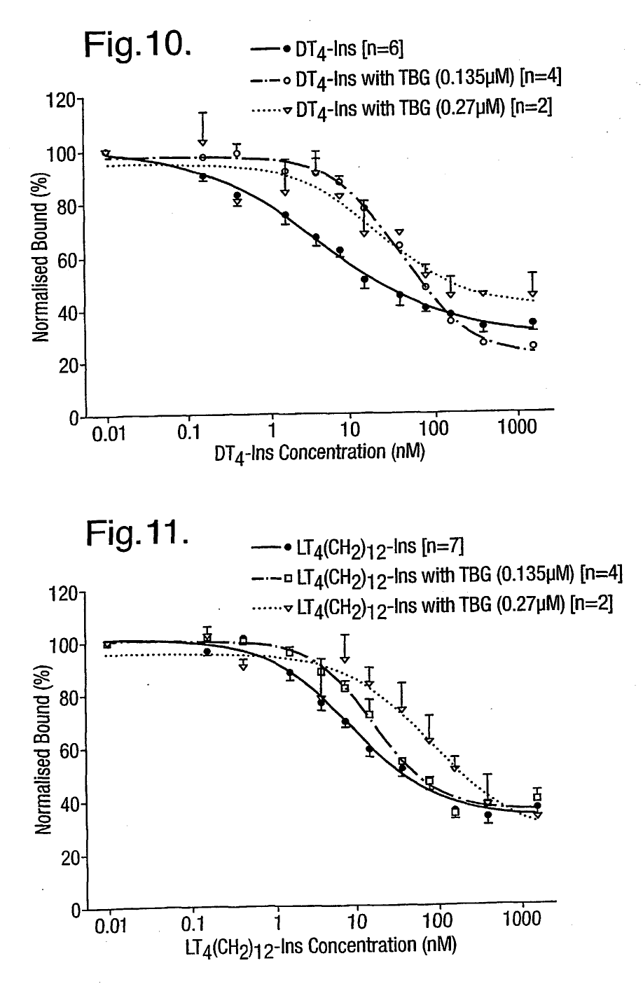

FIG. 10 shows the inhibition of ¹²⁵I-Ins binding to LPM by DT4-ins in the absence of and presence of two different concentrations of TBG. FIG. 11 shows the corresponding curves for LT4(CH₂)₁₂Ins.

As for TBG, addition at 0.135 μM (half physicological concentration) to DT₄Ins caused a non-parallel shift of the binding curve in a similar fashion to that when normal human serum was added. Its ED50 and RPE therefore, cannot be compared to those in the absence of THBPs. There was also no displacement of [¹²⁵I] insulin up till ≈5 nM of DT₄-Ins and the two curves crossed at ≈110 nM. When 0.27 μM TBG was added, the curve was reverted to being parallel to the curve of DT₄-Ins without THBP. The ED50 was significantly higher than DT₄-Ins in the absence of TBG (p<0.05), and the RPE was 25.4% (15.9-37.9%).

For LT₄(CH₂)₁₂-Ins adding 0.135 μM TBG also produced a significantly non-parallel shift of the curve to the right (FIG. 15), hence ED50 and RPE were not valid comparisons. When 0.27 μM TBG was added, the curve was shifted to the right in a parallel fashion. Its ED50 was significantly higher than binding in the absence of THBP (<p0.05), and its RPE was 23.5% (14.2-36.1%).

TABLE 3
Binding of Analogues to THBPs in FPLC

| | Mean % Bound | Mean % Free | |
|---|---|---|---|
| | (fractions 5.5- | (fractions | Significant |
| Analogue and THBP | 15 ml) n = 3 | 15.5-25 ml) | difference* |
| H-Ins | | | |
| 1. Normal human serum | 1.61 ± 0.47 | 98.39 | all others |
| 2. HSA (5% w/v) | 9.02 ± 3.12 | 90.98 | all others |
| 3. TBG | 9.85 ± 2.14 | 90.15 | all others |
| LT4-Ins | | | |
| 4. Normal human serum | 63.22 ± 0.12 | 36.78 | all others |
| 5. HSA (5% w/v) | 72.37 ± 2.31 | 27.63 | 2, 11 |
| 6. TBG | 73.67 ± 3.41 | 26.33 | 3, 9 |
| DT4-Ins | | | |
| 7. Normal human serum | 77.84 ± 2.41 | 22.16 | 1, 4 |
| 8. HSA (5% w/v) | 77.11 ± 1.93 | 22.89 | 2, 11 |
| 9. TBG | 83.97 ± 2.60 | 16.03 | 3, 6, 12 |
| LT4(CH2)12-Ins | | | |
| 10. Normal human serum | 75.60 ± 2.91 | 24.4 | 1, 4 |
| 11. HSA (5% w/v) | 86.32 ± 2.06 | 13.68 | All others |
| 12. TBG | 74.26 ± 1.76 | 25.74 | 3, 9 |

% bound calculated as (total IRI in fractions 5.5-15 ml)/(total IRI in fractions 5.5-25 ml)
% free calculated as (total IRI in fractions 15.5-25 ml)/(total IRI in fractions 5.5-25 ml)
*Significantly different from other Ins with the same THBP (p < 0.05)

TABLE 4
Mean ED50 - Equiblibrium binding tests (LPM)

| Analogue and THBP | Mean ED50 (nM) ± SEM | n |
|---|---|---|
| H-Ins | 8.49 ± 0.69 § | 10 |
| LT₄-Ins | 12.46 ± 0.86 * | 5 |
| DT₄-Ins | 22.23 ± 1.31 *§ | 6 |
| +Normal Human Serum | NC | 5 |
| (1:2.2) | | |
| +HSA (5% w/v) | 26.40 ± 1.01 | 5 |
| +TBG (0.135 μM) | NC | 4 |
| +TBG (0.27 μM) | 108.86 ± 3.78 † | 2 |
| LT₄-(CH₂)₁₂-Ins | 25.13 ± 0.88 *§ | 7 |
| +Normal Human Serum | 89.51 ± 2.03 † | 5 |
| (1:2.2) | | |
| +HSA (5% w/v) | 51.06 ± 1.50 † | 5 |
| +TBG (0.135 μM) | NC | 4 |
| +TBG (0.27 μM) | 113.4 ± 3.69 | 2 |

*Significantly difference (p < 0.05) from H-Ins
§ Significantly different (p < 0.05) from LT₄-Ins
† Significantly different (p < 0.05) from the same analogue without THBP.
NC Non comparable. Binding curve shows significantly non-parallel shift (F < 0.05), as calculated by PARLIN computer software. ED 50 is therefore, not a valid comparison with other curves.

TABLE 5
Relative Potency Estimates - Equilibrium Binding Tests (LPM)

| | Relative Potency | |
|---|---|---|
| Analogue and THBP | Estimates | 95% Fiducial Limits |
| H-Ins | 100% | |
| LT₄-Ins | 63.5% | 40.5-96.7% |
| DT₄-Ins | 45.4% | 27.9-70.0% |
| LT₄-(CH₂)₁₂-Ins | 22.6% | 14.1-33.8% |
| LT₄-Ins | 100% | |
| DT₄-Ins | 68.5% | 42.9-106.4% |
| LT₄-(CH₂)₁₂-Ins | 34.0% | 23.14-48.0% |
| DT₄-Ins | 100% | |
| +Normal Human Serum (1:2.2) | 17.2% | 8.3-28.3% |
| +HSA (5% w/v) | 67.3% | 37.8-115.0% |
| +TBG (0.135 μM) | * | * |
| +TBG (0.27 μM) | 25.4% | 15.9-37.9% |
| LT₄-(CH₂)₁₂-Ins | 100% | |
| +Normal Human Serum (1:2.2) | 21.0% | 11.3-34.5% |
| +HSA (5% w/v) | 92.8% | 66.6-129.2% |
| +TBG (0.135 μM) | * | * |
| +TBG (0.27 μM) | 23.5% | 13.2-36.1% |

All values show insignificant heteroscedasticity (Bartlett _{X}² test, p > 0.05)
* Significant non parallelism (F > 0.05). RPE is therefore non-comparable with others.

1. A compound consisting of insulin or a functional equivalent thereof having covalently bound to the α amine group of the B1 residue a 3,3′,5,5′-tetraiodo-D-thyronyl group (DT4yl).

2. A compound according to claim 1 in which the DT4yl group is bound through a linker.

3. A compound consisting of insulin or a functional equivalent thereof having covalently bound to the α-amine group of its B1 residue an N—C₁₋₄alkanoyl-iodothyronyl group.

4. A compound according to claim 3 in which the iodothyronyl group is an N-alkanoyl-3,3′,5,5′-tetra iodothyronyl group.

5. A compound according to claim 4 in which the iodothyronyl group is a N-alkanoyl 3,3′,5,5′-tetraiodo-D-thyronyl group.

6. A compound according to claim 3 in which the C₁₋₄ alkanoyl group is acetyl.

7. A compound according to claim 3 in which the N-alkanoyl-iodothyronyl group is joined to the α-amine group of the B1 residue through a linker.

8. A compound consisting of insulin or a functional equivalent thereof having covalently bound thereto a thyroid hormone, via a linker which has the general formula —OC—(CR₂)ₙ—NR¹— in which the —OC— is joined to the insulin, the NR¹— is joined to the thyroid hormone, each R is independently selected from H and C₁₋₄ alkyl, and n is an integer of at least 11, R¹ is H, C₁₋₄-alkyl or C₁₋₄-alkanoyl.

9. A compound according to claim 8 in which the —OC group of the linker is joined to the α-amine group of the B1 residue of the insulin or functional equivalent.

10. A compound according to claim 8 in which the thyroid hormone is 3,3′,5,5′-tetraiodothyronine.

11. A compound according to claim 8 in which the linker is —OC—(CH₂)₁₁—NH—.

12. A compound according to claim 10 in which the linker is —OC—(CH₂)₁₁—NH—.

13. A composition comprising a compound according to any preceding claim and a carrier.

14. A pharmaceutical composition comprising a compound according to any of claims 1 to 12 and a pharmaceutical excipient.

15. A compound according to any of claims 1 to 12 for use in a method of treatment of a human or animal by therapy or diagnosis.

16. Use of a compound according to any of claims 1 to 12 in the manufacture of a composition for use in a method of treatment of a human or animal by therapy or diagnosis.

17. Use according to claim 16 in which the method of treatment is insulin replacement therapy.

18. Use according to claim 17 in which the human or animal is diabetic.

19. A method in which free amine group of a peptide is thyronylated by a process comprising the steps:
a) reacting i) a thyronyl reagent of the general formula I 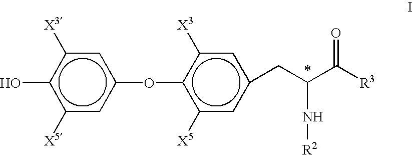
in which each group X³, X^{3′}, X⁵ and X^{5′} is selected from H and I, provided that at elast two of the groups represent I;
R² is an amine protecting group; and
R³is a carboxylic activating group,
with ii) an amine compound ₘ(R⁴N)R⁵(NH₂)ₚ.
in which R⁵ is a (m+p)-functional organic group;
R⁴ is an amine protecting group other than R²;
m is 0 or an integer of up to 10;
p is an integer of at least 1,
b) the protected intermediates treated in a selective amine deprotection step under conditions such that protecting group R² is removed, but any R⁴ groups are not removed, to produce a deprotected intermediate; and
c) the deprotected amine group of the deprotected intermediate is acylated by a C₁₋₄-alkanoyl group in an alkanoylation step to produce an N-alkanoylated compound.

20. Method according to claim 19 in which R² is a tert-butoxy-carbonyl group.

21. Method according to claim 19 in which the or each R⁴ is a methylsulphonylethoxycarbonyl.

22. Method according to claim 19 in which the C₁₋₄ alkanoyl group is an acetyl group.

23. Method according to claim 19 in which m is at least 1 and in which step c) is treated in a second amine deprotection step in which the or each protecting group R⁴ is removed.

24. Method according to claim 19 in which the asymmetric carbon atom C* is in the L-conforiguration.

25. Method according to claim 19 in which the asymmetric carbon atom C* in the D-conforiguration.
